(19) United States
(12) Patent Application Publication
(10) Pub. No.: US 2008/0287684 A1
(43) Pub. Date: Nov. 20, 2008

(54) Method for Producing Ionic Liquids

(75) Inventors: Kai Michael Exner, Mumbai Powai (IN); Klemens Massonne, Bad Durkheim (DE); Veit Stegmann, Mannheim (DE); Matthias Maase, Speyer (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, Ludwigshafen (DE)

(21) Appl. No.: 12/094,411

(22) PCT Filed: Nov. 15, 2006
(86) PCT No.: PCT/EP06/68465

§ 371 Date: Jun. 30, 2008

(30) Foreign Application Priority Data
Nov. 21, 2005 (DE) 10 2005 055 815.1

(51) Int. Cl. C07D 233/54

(52) U.S. Cl. 548/335.1

(57) ABSTRACT

The present invention describes a process for preparing ionic liquids (I) by reacting ionic liquids (II) whose anion is a halide with an acid (III), with the resulting hydrogen halide being scavenged by means of an amine and the resulting ammonium halide being able to be separated off.

The present invention describes a process for preparing ionic liquids (I) by reacting ionic liquids (II) whose anion is a halide with an acid (III), with the resulting hydrogen halide being scavenged by means of an amine and the resulting ammonium halide being able to be separated off.

Ionic liquids are becoming increasing important as solvents, e.g. for carrying out chemical reactions. For example, P. Wasserscheidt, in Chemie in unserer Zeit 37, 52 (2003), gives an overview of the use of ionic liquids in multiphase catalysis. However, other applications have also been proposed, e.g. their use in extraction or separation processes, as heat transfer media, etc.

In all these applications, the purity of the ionic liquids used is important. For example, impurities can have an adverse effect on the course of chemical reactions. Thus, P. Tyson et al. (Electrochemical Society Proceedings, Vol. 99-41, 161) have referred to problems when chloride-comprising ionic liquids are used in liquid-phase hydrogenation and in the Suzuki coupling. The preparation of ionic liquids therefore has to provide ionic liquids having a high purity.

Ionic liquids are frequently prepared by quaternization of the corresponding nitrogen or phosphorus compounds, generally using alkyl halides as alkylating reagents. The ionic liquids obtained in this way can then themselves be used as solvents, etc. However, they also serve as starting material for the preparation of ionic liquids whose anion is not a halide by replacing the anion in a metathesis reaction. However, this frequently suffers from the problem that the resulting ionic liquid still comprises traces of halide which can lead to the above-described problems when the liquid is used as solvent, etc. Otherwise, the removal of the traces of halide is technically complicated, thus incurring high operating costs and/or capital costs and thus having a highly adverse effect on the economics of the process. An example which may be mentioned is the stripping of ionic liquids comprising HCl, since stripping times of sometimes several days have to be reckoned with here.

It is therefore an object of the present invention to provide an industrially useable process for preparing ionic liquids, in particular ionic liquids having a reduced halide content.

It has been found that ionic liquids of the formula (I)

[A]⁺ₙ[Y]ⁿ⁻ (I)

where
[A]⁺ is a quaternary nitrogen heterocycle cation, oxonium cation, sulfonium cation or phosphonium cation;
n is 1, 2, 3 or 4; and
[Y]ⁿ⁻ is an anion selected from among
the group of sulfates, sulfites and sulfonates,
the group of phosphates,
the group of phosphonates and phosphinates,
the group of phosphites,
the group of phosphonites and phosphinites,
the group of carboxylic acids and
NO₃—;
   are obtained by reacting an ionic liquid of the formula (II)

[A]⁺[X]⁻ (II)

where
[A]⁺ is as defined above; and
[X]⁻ is fluoride, chloride, bromide or iodide;
with an acid of the formula III

H⁺ₙ[Y]ⁿ⁻ (III)

where n and [Y]ⁿ⁻ are as defined above;
with the resulting acid of the formula IV

H⁺[X]⁻ (IV)

where [X]⁻ is as defined above; being scavenged by means of an amine of the formula V

NR¹R¹R³ (V)

where
R¹, R² and R³ are each, independently of one another, hydrogen or C₁-C₂₀-alkyl, which may optionally be substituted, with the number of carbon atoms of the radicals R¹, R² and R³ together being at least 10;
   and the resulting ammonium halide of the formula VI

[HNR¹R²R³]⁺[X]⁻ (VI)

where the radicals X, R¹, R² and R³ are as defined above;
being separated off.

The reaction of the ionic liquid of the formula II with the acid of the formula III is usually carried out a temperature of from −50° C. to 150° C., particularly preferably at from −20 to 120° C., in particular from 0 to 100° C., more preferably from 20 to 60° C.

In general, the reaction takes place under atmospheric pressure.

The ionic liquid of the formula II and the acid of the formula III are usually used in the stoichiometric ratio. In some cases, it can also be advantageous to use one or other components in excess.

In general, the amine of the formula V is used in the stoichiometric ratio or in excess relative to the acid of the formula II. In one embodiment, the amine is used in the stoichiometric ratio. In a further embodiment, the amine of the formula V is used in excess, preferably in the range from 1.5 to 2.5 times, in particular from 1.8 to 2.2 times, the stoichiometric ratio. In a further embodiment in which the amine is continuously regenerated, it is also possible to use the amine in a substoichiometric amount, based on a snapshot.

In some cases, it can be advantageous to carry out the process of the invention in bulk.

It is also possible to work in the presence of a solvent. Possible solvents are those in which the ammonium halide of the formula VI dissolves and the ionic liquid of the formula I does not dissolve. Solvents suitable for this purpose are, for example, aromatic hydrocarbons such as benzene, toluene, o-xylene, m-xylene or p-xylene, chlorinated aromatic hydrocarbons such as chlorobenzene or cyclic hydrocarbons such as cyclohexane.

The amine of the formula V is usually selected so that both it and the ammonium halide of the formula VI formed does not dissolve or dissolves only to a slight extent in the ionic liquid of the formula I. The solubility of the amine of the formula V or of the ammonium halide of the formula VI in the ionic liquid of the formula I is in each case preferably less than 10%, particularly preferably less than 5%, in particular less than 2% (expressed as molar ratio of amine or ammonium halide to the ionic liquid). This applies particularly when no solvent is employed.

In a further embodiment which applies particularly to the case where the reaction is carried out in the presence of a solvent, the amine of the formula V is selected so that both it and the ammonium halide of the formula VI formed are more readily soluble in the solvent used than in the ionic liquid of the formula I.

The separation of the reaction products of the formulae I and VI is carried out by liquid-liquid phase separation.
In one embodiment of the present invention, no solvent is employed.
Here, the ionic liquid of the formula II, the acid of the formula III and the amine of the formula V are combined and the mixture is brought to the desired temperature. After the reaction is complete, the phases are separated. One phase comprises the ionic liquid of the formula I and the other phase comprises the ammonium halide of the formula VI formed and possibly unreacted amine of the formula V (if this is used in excess).
In a particular embodiment, the amine of the formula V is added to the reaction mixture in two or more portions, with the phases of the reaction mixture being able to be separated and ammonium halide of the formula VI formed and possibly unreacted amine of the formula V being able to be separated off before renewed addition of amine.
In a further embodiment, the ionic liquid of the formula II and the acid of the formula III are combined. After the reaction is complete, the amine of the formula V is added and after ammonium halide formation is complete the phases are separated again. One phase comprises the ionic liquid of the formula I and the other phase comprises the ammonium halide of the formula VI formed and possibly unreacted amine of the formula V (if this is used in excess). Here, it can be advantageous to bring the initially charged mixture of ionic liquid of the formula II and acid of the formula III to the desired temperature. However, it is also possible to set the desired temperature after addition of the amine of the formula V.
In a particular embodiment, the amine of the formula V is added to the reaction mixture in two or more portions, with the phases of the reaction mixture being separated and ammonium halide of the formula VI formed and possibly unreacted amine of the formula V being separated off before renewed addition of amine.
In a further embodiment, the ionic liquid of the formula II is initially charged and the acid of the formula III and the amine of the formula V are introduced in parallel. After the reaction is complete, the phases are separated. One phase comprises the ionic liquid of the formula I and the other phase comprises the ammonium halide of the formula VI formed and possibly unreacted amine of the formula V (if this is used in excess). Here, it can be advantageous to bring the ionic liquid of the formula II to the desired temperature. However, it is also possible to set the desired temperature after addition of the acid of the formula III and the amine of the formula V.
Here too, it can be advantageous to interrupt the addition, carry out an intermediate phase separation and separate off ammonium halide of the formula VI formed and possibly unreacted amine of the formula V (if this is used in excess).
In a further embodiment of the present invention, the separation of the phases is carried out in the presence of a solvent. Here, the reaction mixtures described in the embodiments without solvents are admixed with a solvent prior to phase separation and the phases are subsequently separated. One phase comprises the ionic liquid of the formula I and the other phase comprises the solvent and the ammonium halide of the formula VI formed and possibly unreacted amine of the formula V (if this is used in excess).
Here, it can be advantageous to add the solvent in two or more portions and separate off the corresponding phases according to the principles of extraction known to those skilled in the art.
In a particular embodiment, the first phase separation is carried out in the absence of a solvent and an extraction with the solvent is subsequently carried out.
In a further embodiment of the present invention, the preparation of the ionic liquid of the formula I is carried out in the presence of a solvent.
Here, the ionic liquid of the formula II, the acid of the formula III, the amine of the formula V and the solvent are combined and the mixture is brought to the desired temperature. After the reaction is complete, the phases are separated. One phase comprises the ionic liquid of the formula I and the other phase comprises the solvent and the ammonium halide of the formula VI formed and possibly unreacted amine of the formula V (if this is used in excess).
In a particular embodiment, only part of the solvent is added to the starting materials and the other part is used for the removal of the ammonium halide of the formula VI and possibly the unreacted amine of the formula V (if this is used in excess).
In a further particular embodiment, the amine of the formula V is added to the reaction mixture in two or more portions, if appropriate in the solvent, with the phases of the reaction mixture being separated and the solvent and the ammonium halide of the formula VI formed and possibly unreacted amine of the formula V being separated off before renewed addition of amine.
In a further embodiment, the ionic liquid of the formula II and the acid of the formula III are combined. After the reaction is complete, the amine of the formula V and the solvent are added and after the ammonium halide formation is complete the phases are separated again. One phase comprises the ionic liquid of the formula I and the other phase comprises the solvent and the ammonium halide of the formula VI formed and possibly unreacted amine of the formula V (if this is used in excess). Here, it can be advantageous to bring the initially charged mixture of ionic liquid of the formula II and acid of the formula III to the desired temperature. However, it is also possible to set the desired temperature after addition of the amine of the formula V.
In a particular embodiment, only part of the solvent is added to the amine of the formula V and the other part is used for the removal of the ammonium halide of the formula VI and possibly the unreacted amine of the formula V (if this is used in excess).
In a further particular embodiment, the amine of the formula V is added to the reaction mixture in two or more portions, with the phases of the reaction mixture being separated and ammonium halide of the formula VI formed and possibly unreacted amine of the formula V being separated off before renewed addition of amine.
In a further embodiment, the ionic liquid of the formula II is initially charged and the acid of the formula III and the amine of the formula V are introduced in parallel. Here, the acid of the formula III and/or the amine of the formula V are added in the solvent. After the reaction is complete, the phases are separated.
One phase comprises the ionic liquid of the formula I and the other phase comprises the solvent and the ammonium halide of the formula VI formed and possibly unreacted amine of the formula V (if this is used in excess). Here, it can be advantageous to bring the ionic liquid of the formula II to the desired temperature. However, it is also possible to set the desired temperature after addition of the acid of the formula III and the amine of the formula V.
In a particular embodiment, only part of the solvent is added to the acid of the formula III and/or the amine of the formula V and the other part is used for the removal of the ammonium halide of the formula VI and possibly the unreacted amine of the formula V (if this is used in excess).
Here too, it can be advantageous to interrupt the addition, carry out an intermediate phase separation and separate off ammonium halide of the formula VI formed and possibly unreacted amine of the formula V (if this is used in excess).

The preparation of the ionic liquid of the formula I can be carried out batchwise, continuously or by a semibatch process. The removal of the ammonium halide of the formula VI formed is adapted accordingly.

The liquid-liquid phase separation is carried out, for example, by means of techniques as are described in Ullmann's Encyclopedia of Industrial Chemistry, sixth edition, 2000 electronic release, chapter “Liquid-Liquid Extraction”, particularly in subchapter 4 “Phase-Separation Equipment”; preferably by means of decantation, phase separators, centrifugation or mixer-settler apparatuses, particularly preferably by means of phase separators.

Furthermore, it may be advantageous to recover the original amine of the formula V from the ammonium halide of the formula VI formed and, if desired, use it again in the process of the invention for preparing the ionic liquids of the formula I.

The recovery of the amines of the formula V can, for example, be effected by treating the ammonium halide of the formula VI with a strong base, e.g. NaOH, KOH, Ca(OH)₂, milk of lime, Na₂CO₃, NaHCO₃, K₂CO₃ or KHCO₃, if appropriate in a solvent such as water, methanol, ethanol, n-propanol or isopropanol, n-butanol, n-pentanol or butanol or pentanol isomer mixtures or acetone.

In a preferred embodiment of the invention, the strong base is used in a very concentrated solution, particularly preferably an aqueous solution, for example a solution having a concentration of at least 5% by weight, preferably at least 10% by weight and particularly preferably at least 15% by weight.

The amount of base is usually selected so as to correspond to the stoichiometry. In some cases, it can be advantageous to use a substoichiometric amount or an excess. An equimolar amount is generally employed.

If the preparation of the ionic liquid of the formula I has been carried out in the absence of a solvent, the amine of the formula V can, if it forms a separate phase, be separated off or can otherwise be removed from the mixture by distillation. If necessary, the amine of the formula V can be separated off by extraction with an extractant. Possible extractants are customary solvents such as aliphatic ethers, e.g. diethyl ether or methyl tert-butyl ether, cyclic ethers such as tetrahydrofuran, 1,3-dioxane or 1,4-dioxane, hydrocarbons such as pentane, hexane, cyclopentane or cyclohexane, aromatic hydrocarbons such as benzene, toluene, o-xylene, m-xylene or p-xylene, chlorinated hydrocarbons such as methylene chloride, chloroform or 1,2-dichloroethane or chlorinated aromatic hydrocarbons such as chlorobenzene.

If necessary, the amine of the formula V can be purified further, e.g. by distillation, before reuse.

If a solvent is used in the preparation of the ionic liquid of the formula I, the amine of the formula V can be recovered by treating the solution of the ammonium halide of the formula VI with the abovementioned bases and extracting the liberated amine of the formula V with the appropriate solvent in a manner known to those skilled in the art and isolating the amine of the formula V by customary methods, e.g. distillation.

Purification can be effected by, for example, single or multiple washing, drying, filtration, stripping, distillation and/or rectification.

Drying can be carried out, for example, by removal of any water present by means of distillation or azeotropic distillation with benzene, toluene, xylene, butanol or cyclohexane.

A filtration can be useful, for example, to remove precipitated solids or to eliminate discoloration which may have occurred, for example by filtration through activated carbon, aluminum oxide, Celite or silica gel.

A distillation, for example to separate off any solvent comprised, can preferably be carried out by means of a falling film or thin film evaporator, if appropriate under reduced pressure, with a column being able to be superposed to improve the separation.

Correspondingly, the solvent used can if necessary be purified and, if desired, be reused.

The present invention thus further provides a process for preparing ionic liquids of the formula I, which comprises the following steps:

reaction of an ionic liquid of the formula II with an acid of the formula III;
scavenging of the resulting acid of the formula IV by means of an amine of the formula V;
separation of the resulting ionic liquid of the formula I and the resulting ammonium halide of the formula VI by liquid/liquid phase separation;
addition of a base to the phase which comprises the ammonium halide of the formula VI formed and liberation of the amine of the formula V;
if appropriate, purification of the amine of the formula V obtained in crude form; and,
if appropriate, recirculation of the optionally purified amine to the reaction with an anionic liquid of the formula II with an acid of the formula III.

In a preferred embodiment, the ionic liquid of the formula II and the acid of the formula III are initially charged and the mixture is extracted continuously with the amine of the formula V, with the free amine being continuously regenerated from the ammonium halide which has been separated off or the ammonium halide/amine and is recirculated to the extraction process. Here, the amine can be used in a substoichiometric molar amount relative to the free acids, based on a snapshot of the extraction section. The extraction can be aided by a solvent or be carried out fully continuously, with amine phase and the phase comprising the ionic liquid being conveyed past one another continuously.

The ionic liquids of the formulae I and II and also the ammonium halide of the formula VI preferably have, independently of one another, a melting point of less than 180° C. Furthermore, the melting point is in the range from −50° C. to 150° C., preferably in the range from −20° C. to 120° C. and preferably below 100° C.

Preference is given to preparing ionic liquids of the formula I which have a molecular weight of less than 1000 g/mol, preferably less than 500 g/mol, in particular less than 250 g/mol.

Preference is given to preparing ionic liquids of the formula I in which the variables, either alone or in combination, have the following meanings:

[A]+ is a cation selected from among the compounds of the formula (Ia) to (Iv),

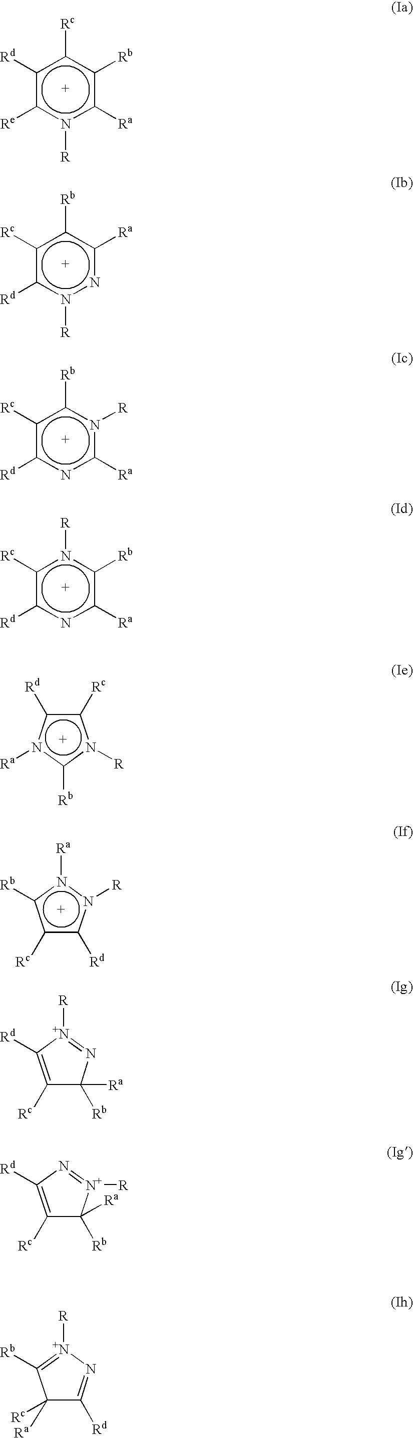

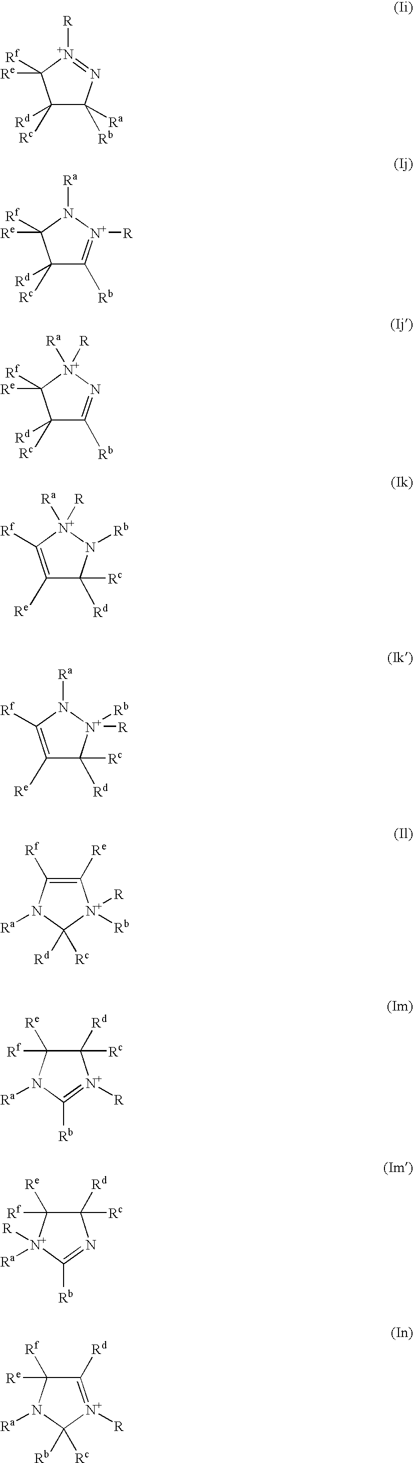

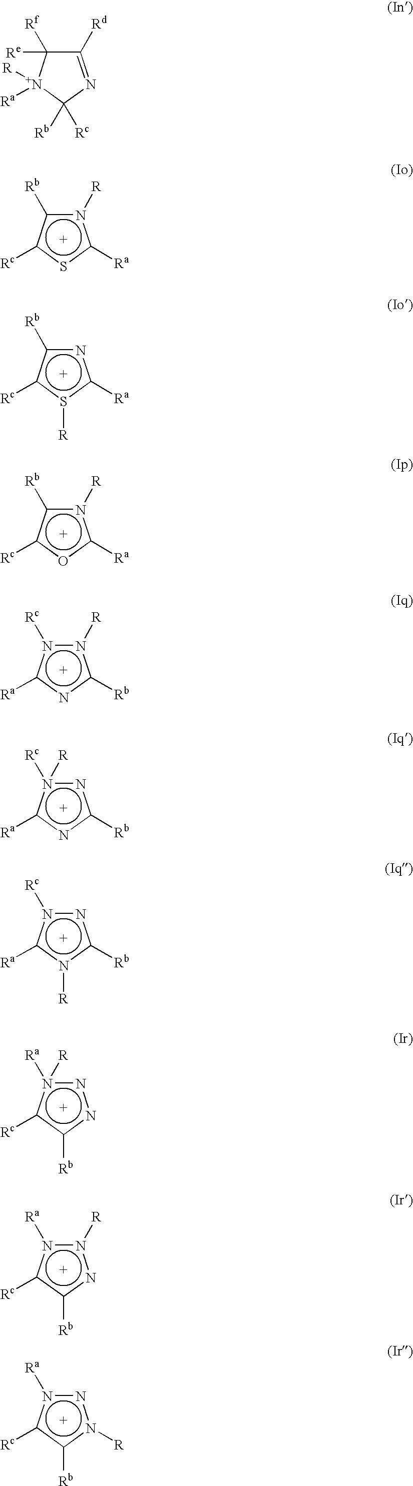

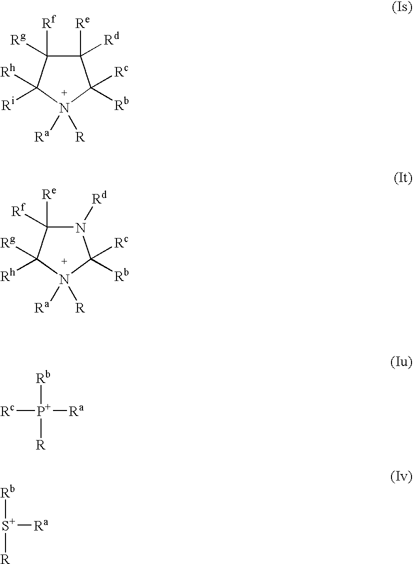

In the above formulae (Ia) to (Iv):
the radical R is hydrogen or a carbon-comprising organic, saturated or unsaturated, acyclic or cyclic, aliphatic, aromatic or araliphatic radical which has from 1 to 20 carbon atoms and may be unsubstituted or be interrupted or substituted by from 1 to 5 heteroatoms or functional groups; and
the radicals R^{a} to Rⁱ are each, independently of one another, hydrogen or a carbon-comprising organic, saturated or unsaturated, acyclic or cyclic, aliphatic, aromatic or araliphatic radical which has from 1 to 20 carbon atoms and may be unsubstituted or be interrupted or substituted by from 1 to 5 heteroatoms or functional groups, where the radicals R^{a} to Rⁱ which are bound to a carbon atom (and not to a heteroatom) in the formulae (I) above can additionally be halogen or a functional group; or
two adjacent radicals from the group consisting of R^{a} to Rⁱ may together also form a divalent, carbon-comprising organic, saturated or unsaturated, acyclic or cyclic, aliphatic, aromatic or araliphatic radical which has from 1 to 30 carbon atoms and may be unsubstituted or be interrupted or substituted by from 1 to 5 heteroatoms or functional groups.

In the definitions of the radicals R and R^{a} to Rⁱ, possible heteroatoms are in principle all heteroatoms which are able to formally replace a —CH₂— group, a —CH═ group, a —C≡ group or a ═C═ group. If the hydrocarbon-comprising radical comprises heteroatoms, then oxygen, nitrogen, sulfur, phosphorus and silicon are preferred. Preferred groups are, in particular, —O—, —S—, —SO—, —SO₂—, —NR′—, —N═, —PR′—, —PR′₂ and —SiR′₂—, where the radicals R′ are the remaining part of the carbon-comprising radical. In the cases in which the radicals R^{a} to Rⁱ are bound to a carbon atom (and not a heteroatom) in the abovementioned formula (I), they can also be bound directly via the heteroatom.

Suitable functional groups are in principle all functional groups which can be bound to a carbon atom or a heteroatom. Suitable examples are —OH (hydroxy), ═O (in particular as carbonyl group), —NH₂ (amino), ═NH (imino), —COOH (carboxy), —CONH₂ (carboxamide), —SO₃H (sulfo) and —CN (cyano). Functional groups and heteroatoms can also be directly adjacent, so that combinations of a plurality of adjacent atoms, for instance —O— (ether), —S-(thioether), —COO— (ester), —CONH— (secondary amide) or —CONR′— (tertiary amide), are also comprised, for example di-(C₁-C₄-alkyl)amino, C₁-C₄-alkyloxycarbonyl or C₁-C₄-alkyloxy.

As halogens, mention may be made of fluorine, chlorine, bromine and iodine.

The radical R is preferably
unbranched or branched C₁-C₁₈-alkyl which may be unsubstituted or substituted by one or more hydroxy, halogen, phenyl, cyano, C₁-C₆-alkoxycarbonyl and/or sulfonic acid groups and has a total of from 1 to 20 carbon atoms, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl, 1-octadecyl, 2-hydroxyethyl, benzyl, 3-phenylpropyl, 2-cyanoethyl, 2-(methoxycarbonyl)ethyl, 2-(ethoxycarbonyl)ethyl, 2-(n-butoxycarbonyl)ethyl, trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluorisopentyl, 6-hydroxyhexyl and propylsulfonic acid;
glycols, butylene glycols and oligomers thereof having from 1 to 100 units and a hydrogen or a C₁-C₈-alkyl as end group, for example R^{A}O—(CHR^{B}—CH₂—O)ₘ—CHR^{B}—CH₂— or R^{A}O—(CH₂CH₂CH₂CH₂O)ₘ—CH₂CH₂CH₂CH₂O— where R^{A} and R^{B} are each preferably hydrogen, methyl or ethyl and m is preferably 0, 1, 2 or 3, in particular 3-oxabutyl, 3-oxapentyl, 3,6-dioxaheptyl, 3,6-dioxaoctyl, 3,6,9-trioxadecyl, 3,6,9-trioxaundecyl, 3,6,9,12-tetraoxamidecyl and 3,6,9,12-tetraoxatetradecyl;
vinyl; and
N,N-di-C₁-C₆-alkylamino such as N,N-dimethylamino and N,N-diethylamino.

The radical R is particularly preferably unbranched and unsubstituted C₁-C₁₈-alkyl, such as methyl, ethyl, 1-propyl, 1-butyl, 1-pentyl, 1-hexyl, 1-heptyl, 1-octyl, 1-decyl, 1-dodecyl, 1-tetradecyl, 1-hexadecyl, 1-octadecyl, in particular methyl, ethyl, 1-butyl and 1-octyl, or CH₃O—(CH₂CH₂O)ₘ—CH₂CH₂— and CH₃CH₂O—(CH₂CH₂O)ₘ—CH₂CH₂— where m is 0, 1, 2 or 3.

Preference is given to the radicals R^{a} to Rⁱ each being, independently of one another,
hydrogen;
halogen;
a functional group;

C₁-C₁₈-alkyl which may optionally be substituted by functional groups, aryl, alkyl, aryloxy, alkyloxy, halogen, heteroatoms and/or heterocycles and/or be interrupted by one or more oxygen and/or sulfur atoms and/or one or more substituted or unsubstituted imino groups;

C₂-C₁₈-alkenyl, which may optionally be substituted by functional groups, aryl, alkyl, aryloxy, alkyloxy, halogen, heteroatoms and/or heterocycles and/or be interrupted by one or more oxygen and/or; sulfur atoms and/or one or more substituted or unsubstituted imino groups;
C₆-C₁₂-aryl which may optionally be substituted by functional groups, aryl, alkyl, aryloxy, alkyloxy, halogen, heteroatoms and/or heterocycles;
C₅-C₁₂-cycloalkyl which may optionally be substituted by functional groups, aryl, alkyl, aryloxy, alkyloxy, halogen, heteroatoms and/or heterocycles;
C₅-C₁₂-cycloalkenyl which may optionally be substituted by functional groups, aryl, alkyl, aryloxy, alkyloxy, halogen, heteroatoms and/or heterocycles; or
a five- or six-membered, oxygen-, nitrogen- and/or sulfur-comprising heterocycle which may optionally be substituted by functional groups, aryl, alkyl, aryloxy, alkyloxy, halogen, heteroatoms and/or heterocycles; or
   two adjacent radicals together form an unsaturated, saturated or aromatic ring which may optionally be substituted by functional groups, aryl, alkyl, aryloxy, alkyloxy, halogen, heteroatoms and/or heterocycles and may optionally be interrupted by one or more oxygen and/or sulfur atoms and/or one or more substituted or unsubstituted imino groups.

C₁-C₁₈-alkyl which may optionally be substituted by functional groups, aryl, alkyl, aryloxy, alkyloxy, halogen, heteroatoms and/or heterocycles is preferably methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, 2-ethylhexyl, 2,4,4-trimethylpentyl, 1,1,3,3-tetramethylbutyl, 1-nonyl, 1-decyl, 1-undecyl, 1-dodecyl, 1-tridecyl, 1-tetradecyl, 1-pentadecyl, 1-hexadecyl, 1-heptadecyl, 1-octadecyl, cyclopentylmethyl, 2-cyclopentylethyl, 3-cyclopentylpropyl, cyclohexylmethyl, 2-cyclohexylethyl, 3-cyclohexylpropyl, benzyl (phenylmethyl), diphenylmethyl (benzhydryl), triphenylmethyl, 1-phenylethyl, 2-phenylethyl, 3-phenylpropyl, α,α-dimethylbenzyl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di-(methoxycarbonyl)ethyl, methoxy, ethoxy, formyl, 1,3-dioxolan-2-yl, 1,3-dioxan-2-yl, 2-methyl-1,3-dioxolan-2-yl, 4-methyl-1,3-dioxolan-2-yl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl, 6-ethoxyhexyl, acetyl, CₘF_{2(m-a)+(1-b)}H_{2a+b} where m is from 1 to 30, 0≦a≦m and b=0 or 1 (for example CF₃, C₂F₅, CH₂CH₂—C₍ₘ₋₂₎F₂₍ₘ₋₂₎₊₁, C₆F₁₃, C₈F₁₇, C₁₀F₂₁, C₁₂F₂₅), chloromethyl, 2-chloroethyl, trichloromethyl, 1,1-dimethyl-2-chloroethyl, methoxymethyl, 2-butoxyethyl, diethoxymethyl, diethoxyethyl, 2-isopropoxyethyl, 2-butoxypropyl, 2-octyloxyethyl, 2-methoxyisopropyl, 2-(methoxycarbonyl)ethyl, 2-(ethoxycarbonyl)ethyl, 2-(n-butoxycarbonyl)ethyl, butylthiomethyl, 2-dodecylthioethyl, 2-phenylthioethyl, 5-hydroxy-3-oxapentyl, 8-hydroxy-3,6-dioxaoctyl, 11-hydroxy-3,6,9-trioxaundecyl, 7-hydroxy-4-oxaheptyl,11-hydroxy-4,8-dioxaundecyl, 15-hydroxy-4,8,12-trioxapentadecyl, 9-hydroxy-5-oxanonyl, 14-Hydroxy-5,10-dioxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-dioxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl.

C₂-C₁₈-alkenyl which may optionally be substituted by functional groups, aryl, alkyl, aryloxy, alkyloxy, halogen, heteroatoms and/or heterocycles and/or be interrupted by one or more oxygen and/or sulfur atoms and/or one or more substituted or unsubstituted imino groups is preferably vinyl, 2-propenyl, 3-butenyl, cis-2-butenyl, trans-2-butenyl or CₘF_{2(m-a)-(1-b)}H_{2a-b} where m≦30, 0≦a≦m and b=0 or 1.

C₆-C₁₂-aryl which may optionally be substituted by functional groups, aryl, alkyl, aryloxy, alkyloxy, halogen, heteroatoms and/or heterocycles is preferably phenyl, tolyl, xylyl, α-naphthyl, β-naphthyl, 4-diphenylyl, chlorophenyl, dichlorophenyl, trichloro-phenyl, difluorophenyl, methylphenyl, dimethylphenyl, trimethylphenyl, ethylphenyl, diethylphenyl, iso-propylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2-nitrophenyl, 4-nitrophenyl, 2,4-dinitrophenyl, 2,6-dinitrophenyl, 4-dimethylaminophenyl, 4-acetylphenyl, methoxyethylphenyl, ethoxymethylphenyl, methylthiophenyl, isopropylthiophenyl or tert-butylthiophenyl or C₆F₍₅₋ₐ₎Hₐ where 0≦a≦5.

C₅-C₁₂-cycloalkyl which may optionally be substituted by functional groups, aryl, alkyl, aryloxy, alkyloxy, halogen, heteroatoms and/or heterocycles is preferably cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl, CₘF_{2(m- a)-(1-b)}H_{2a-b} where m≦30, 0≦a≦n and b=0 or 1, or a saturated or unsaturated bicyclic system such as norbornyl or norbornenyl.

C₅- to C₁₂-cycloalkenyl which may optionally be substituted by functional groups, aryl, alkyl, aryloxy, alkyloxy, halogen, heteroatoms and/or heterocycles is preferably 3-cyclopentenyl, 2-cyclohexenyl, 3-cyclohexenyl, 2,5-cyclohexadienyl or CₘF_{2(m-a)-3(1- b)}H_{2a-3b} where m≦30, 0≦a≦m and b=0 or 1.

A five- or six-membered, oxygen-, nitrogen- and/or sulfur-comprising heterocycle which may optionally be substituted by functional groups, aryl, alkyl, aryloxy, alkyloxy, halogen, heteroatom and/or heterocycles is preferably furyl, thiophenyl, pyrryl, pyridyl, indolyl, benzoxazolyl, dioxolyl, dioxyl, benzimidazolyl, benzthiazolyl, dimethylpyridyl, methylquinolyl, dimethylpyrryl, methoxyfuryl, dimethoxypyridyl or difluoropyridyl.

If two adjacent radicals together form an unsaturated, saturated or aromatic ring which may optionally be substituted by functional groups, aryl, alkyl, aryloxy, alkyloxy, halogen, heteroatoms and/or heterocycles and may optionally be interrupted by one or more oxygen and/or sulfur atoms and/or one or more substituted or unsubstituted imino groups, they preferably form 1,3-propylene, 1,4-butylene, 1,5-pentylene, 2-oxa-1,3-propylene, 1-oxa-1,3-propylene, 2-oxa-1,3-propylene, 1-oxa-1,3-propenylene, 3-oxa-1,5-pentylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

If the abovementioned radicals comprise oxygen and/or sulfur atoms and/or substituted or unsubstituted imino groups, the number of oxygen and/or sulfur atoms and/or imino groups is not subject to any restrictions. In general, there will be no more than 5 in the radical, preferably no more than 4 and very particularly preferably no more than 3.

If the abovementioned radicals comprise heteroatoms, there is generally at least one carbon atom, preferably at least two carbon atoms, between any two heteroatoms.

Particular preference is given to the radicals R^{a} to Rⁱ each being, independently of one another,
hydrogen;
unbranched or branched C₁-C₁₈-alkyl which may be unsubstituted or substituted by one or more hydroxy, halogen, phenyl, cyano, C₁-C₆-alkylcarbonyl and/or sulfonic acid groups and has a total of from 1 to 20 carbon atoms, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl, 1-octadecyl, 2-hydroxyethyl, benzyl, 3-phenylpropyl, 2-cyanoethyl, 2-(methoxycarbonyl)ethyl, 2-(ethoxycarbonyl)ethyl, 2-(n-butoxy-carbonyl)ethyl, trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl, 6-hydroxyhexyl and propylsulfonic acid;
glycols, butylene glycols and oligomers thereof having from 1 to 100 units and a hydrogen or a C₁-C₈-alkyl as end group, for example R^{A}O—(CHR^{B}—CH₂—O)ₘ—CHR^{B}—CH₂— or R^{A}O—(CH₂CH₂CH₂CH₂O)ₘ—CH₂CH₂CH₂CH₂O— where R^{A} and R^{B} are each preferably hydrogen, methyl or ethyl and m is preferably 0, 1, 2 or 3, in particular 3-oxabutyl, 3-oxapentyl, 3,6-dioxaheptyl, 3,6-dioxaoctyl, 3,6,9-trioxadecyl, 3,6,9-trioxaundecyl, 3,6,9,12-tetraoxamidecyl and 3,6,9,12-tetraoxatetradecyl;
vinyl; and
N,N-di-C₁-C₆-alkylamino, such as N,N-dimethylamino and N,N-diethylamino.

Very particular preference is given to the radicals R^{a} to Rⁱ each being, independently of one another, hydrogen or C₁-C₁₈-alkyl such as methyl, ethyl, 1-butyl, 1-pentyl, 1-hexyl, 1-heptyl, 1-octyl, phenyl, 2-hydroxyethyl, 2-cyanoethyl, 2-(methoxycarbonyl)ethyl, 2-(ethoxycarbonyl)ethyl, 2-(n-butoxycarbonyl)ethyl, N,N-dimethylamino, N,N-diethylamino, chlorine or CH₃O—(CH₂CH₂O)ₘ—CH₂CH₂— and CH₃CH₂O—(CH₂CH₂O)ₘ—CH₂CH₂— where m is 0, 1, 2 or 3.

Very particularly preferred pyridinium ions (Ia) are those in which
one of the radicals R^{a} to R^{e} is methyl, ethyl or chlorine and the remaining radicals R^{a} to R^{e} are each hydrogen;
R^{c} is dimethylamino and the remaining radicals R^{a}, R^{b}, R^{d} and R^{e} are each hydrogen;
all radicals R^{a} to R^{e} are hydrogen;
R^{b} is carboxy or carboxamide and the remaining radicals R^{a}, R^{b}, R^{d} and R^{e} are each hydrogen; or
R^{a} and R^{b} or R^{b} and R^{c} are together 1,4-buta-1,3-dienylene and the remaining radicals R^{a}, R^{b}, R^{c} and R^{d} are each hydrogen;
   and in particular those in which
R^{a} to R^{d} are each hydrogen; or
one of the radicals R^{a} to R^{e} is methyl or ethyl and the remaining radicals R^{a} to R^{e} are each hydrogen.

As very particularly preferred pyridinium ions (IVa), mention may be made of 1-methylpyridinium, 1-ethylpyridinium, 1-(1-butyl)pyridinium, 1-(1-hexyl)pyridinium, 1-(1-octyl)pyridinium, 1-(1-hexyl)pyridinium, 1-(1-octyl)pyridinium, 1-(1-dodecyl)pyridinium, 1-(1-tetradecyl)pyridinium, 1-(1-hexadecyl)pyridinium, 1,2-dimethylpyridinium, 1-ethyl-2-methylpyridinium, 1-(1-butyl)-2-methylpyridinium, 1-(1-hexyl)-2-methylpyridinium, 1-(1-octyl)-2-methylpyridinium, 1-(1-dodecyl)-2-methylpyridinium, 1-(1-tetradecyl)-2-methylpyridinium, 1-(1-hexadecyl)-2-methylpyridinium, 1-methyl-2-ethylpyridinium, 1,2-diethylpyridinium, 1-(1-butyl)-2-ethylpyridinium, 1-(1-hexyl)-2-ethylpyridinium, 1-(1-octyl)-2-ethylpyridinium, 1-(1-dodecyl)-2-ethylpyridinium, 1-(1-tetradecyl)-2-ethylpyridinium, 1-(1-hexadecyl)-2-ethylpyridinium, 1,2-dimethyl-5-ethylpyridinium, 1,5-diethyl-2-methylpyridinium, 1-(1-butyl)-2-methyl-3-ethylpyridinium, 1-(1-hexyl)-2-methyl-3-ethylpyridinium and 1-(1-octyl)-2-methyl-3-ethyl-pyridinium, 1-(1-dodecyl)-2-methyl-3-ethylpyridinium, 1-(1-tetradecyl)-2-methyl-3-ethylpyridinium and 1-(1-hexadecyl)-2-methyl-3-ethylpyridinium.

Very particularly preferred pyridazinium ions (Ib) are those in which
R^{a} to R^{d} are each hydrogen; or
one of the radicals R^{a} to R^{d} is methyl or ethyl and the remaining radicals R^{a} to R^{d} are each hydrogen.

Very particularly preferred pyrimidinium ions (Ic) are those in which
R^{a} is hydrogen, methyl or ethyl and R^{b} to R^{d} are each, independently of one another, hydrogen or methyl; or
R^{a} is hydrogen, methyl or ethyl, R^{b} and R^{d} are each methyl and R^{c} is hydrogen.

Very particularly preferred pyrazinium ions (Id) are those in which
R^{a} is hydrogen, methyl or ethyl and R^{b} to R^{d} are each, independently of one another, hydrogen or methyl;
R^{a} is hydrogen, methyl or ethyl, R^{b} and R^{d} are each methyl and R^{c} is hydrogen;
R^{a} to R^{d} are each methyl; or
R^{a} to R^{d} are each methyl or hydrogen.

Very particularly preferred imidazolium ions (Ie) are those in which
R^{a} is hydrogen, methyl, ethyl, 1-propyl, 1-butyl, 1-pentyl, 1-hexyl, 1-octyl, 2-hydroxyethyl or 2-cyanoethyl and R^{b} to R^{d} are each, independently of one another, hydrogen, methyl or ethyl.

As very particularly preferred imidazolium ions (IVe), mention may be made of 1-methylimidazolium, 1-ethylimidazolium, 1-(1-butyl)imidazolium, 1-(1-octyl)imidazolium, 1-(1-dodecyl)imidazolium, 1-(1-tetradecyl)imidazolium, 1-(1-hexadecyl)imidazolium, 1,3-dimethylimidazolium, 1-ethyl-3-methylimidazolium, 1-(1-butyl)-3-methylimidazolium, 1-(1-butyl)-3-ethylimidazolium, 1-(1-hexyl)-3-methylimidazolium, 1-(1-hexyl)-3-ethylimidazolium, 1-(1-hexyl)-3-butylimidazolium, 1-(1-octyl)-3-methylimidazolium, 1-(1-octyl)-3-ethylimidazolium, 1-(1-octyl)-3-butylimidazolium, 1-(1-dodecyl)-3-methylimidazolium, 1-(1-dodecyl)-3-ethylimidazolium, 1-(1-dodecyl)-3-butylimidazolium, 1-(1-dodecyl)-3-octylimidazolium, 1-(1-tetradecyl)-3-methylimidazolium, 1-(1-tetradecyl)-3-ethylimidazolium, 1-(1-tetradecyl)-3-butylimidazolium, 1-(1-tetradecyl)-3-octylimidazolium, 1-(1-hexadecyl)-3-methylimidazolium, 1-(1-hexadecyl)-3-ethylimidazolium, 1-(1-hexadecyl)-3-butylimidazolium, 1-(1-hexadecyl)-3-octylimidazolium, 1,2-dimethylimidazolium, 1,2,3-trimethylimidazolium, 1-ethyl-2,3-dimethylimidazolium, 1-(1-butyl)-2,3-dimethylimidazolium, 1-(1-hexyl)-2,3-dimethylimidazolium, 1-(1-octyl)-2,3-dimethylimidazolium, 1,4-dimethylimidazolium, 1,3,4-trimethylimidazolium, 1,4-dimethyl-3-ethylimidazolium, 3-butylimidazolium, 1,4-dimethyl-3-octylimidazolium, 1,4,5-trimethylimidazolium, 1,3,4,5-tetramethylimidazolium, 1,4,5-trimethyl-3-ethylimidazolium, 1,4,5-trimethyl-3-butylimidazolium and 1,4,5-trimethyl-3-octylimidazolium.

Very particularly preferred pyrazolium ions (If), (Ig) and (Ig′) are those in which
R^{a} is hydrogen, methyl or ethyl and R^{b} to R^{d} are each, independently of one another, hydrogen or methyl.

Very particularly preferred pyrazolium ions (Ih) are those in which
R^{a} to R^{d} are each, independently of one another, hydrogen or methyl.

Very particularly preferred 1-pyrazolinium ions (Ii) are those in which
R^{a} to R^{f} are each, independently of one another, hydrogen or methyl.

Very particularly preferred 2-pyrazolinium ions (Ij) and (Ij′) are those in which
R^{a} is hydrogen, methyl, ethyl or phenyl and R^{b} to R^{f} are each, independently of one another, hydrogen or methyl.

Very particularly preferred 3-pyrazolinium ions (Ik) and (Ik′) are those in which
R^{a} and R^{b} are each, independently of one another, hydrogen, methyl, ethyl or phenyl and R^{c} to R^{d} are each, independently of one another, hydrogen or methyl.

Very particularly preferred imidazolinium ions (II) are those in which
R^{a} and R^{b} are each, independently of one another, hydrogen, methyl, ethyl, 1-butyl or phenyl, R^{c} and R^{d} are each, independently of one another, hydrogen, methyl or ethyl and R^{e} and R^{f} are each, independently of one another, hydrogen or methyl.

Very particularly preferred imidazolinium ions (Im) and (Im′) are those in which
R^{a} and R^{b} are each, independently of one another, hydrogen, methyl or ethyl and R^{c} to R^{f} are each, independently of one another, hydrogen or methyl.

Very particularly preferred imidazolinium ions (In) and (In′) are those in which
R^{a} to R^{c} are each, independently of one another, hydrogen, methyl or ethyl and R^{d} to R^{f} are each, independently of one another, hydrogen or methyl.

Very particularly preferred thiazolium ions (Io) and (Io′) and oxazolium ions (Ip) are those in which
R^{a} is hydrogen, methyl, ethyl or phenyl and R^{b} and R^{c} are each, independently of one another, hydrogen or methyl.

Very particularly preferred 1,2,4-triazolium ions (Iq), (Iq′) and (Iq″) are those in which
R^{a} and R^{b} are each, independently of one another, hydrogen, methyl, ethyl or phenyl and R^{c} is hydrogen, methyl or phenyl.

Very particularly preferred 1,2,3-triazolium ions (Ir), (Ir′) and (Ir″) are those in which
R^{a} is hydrogen, methyl or ethyl and R^{b} and R^{c} are each, independently of one another, hydrogen or methyl or R^{b} and R^{c} are together 1,4-buta-1,3-dienylene.

Very particularly preferred pyrrolidinium ions (Is) are those in which
R^{a} is hydrogen, methyl, ethyl or phenyl and R^{b} to Rⁱ are each, independently of one another, hydrogen or methyl.

Very particularly preferred imidazolidinium ions (It) are those in which
R^{a} and R^{d} are each, independently of one another, hydrogen, methyl, ethyl or phenyl and R^{b} and R^{c} and also R^{e} to R^{h} are each, independently of one another, hydrogen or methyl.

Very particularly preferred phosphonium ions (Iu) are those in which
R^{a} to R^{c} are each, independently of one another, C₁-C₁₈-alkyl, in particular butyl, isobutyl, 1-hexyl or 1-octyl.

Among the abovementioned heterocyclic cations, preference is given to the pyridinium ions, pyrazolinium ions, pyrazolium ions and the imidazolinium ions and the imidazolium ions.

Particular preference is given to 1-methylpyridinium, 1-ethylpyridinium, 1-(1-butyl)pyridinium, 1-(1-hexyl)pyridinium, 1-(1-octyl)pyridinium, 1-(1-hexyl)pyridinium, 1-(1-octyl)pyridinium, 1-(1-dodecyl)pyridinium, 1-(1-tetradecyl)pyridinium, 1-(1-hexadecyl)pyridinium, 1,2-dimethylpyridinium, 1-ethyl-2-methylpyridinium, 1-(1-butyl)-2-methylpyridinium, 1-(1-hexyl)-2-methylpyridinium, 1-(1-octyl)-2-methylpyridinium, 1-(1-dodecyl)-2-methylpyridinium, 1-(1-tetradecyl)-2-methylpyridinium, 1-(1-hexadecyl)-2-methylpyridinium, 1-methyl-2-ethylpyridinium, 1,2-diethylpyridinium, 1-(1-butyl)-2-ethylpyridinium, 1-(1-hexyl)-2-ethylpyridinium, 1-(1-octyl)-2-ethylpyridinium, 1-(1-dodecyl)-2-ethylpyridinium, 1-(1-tetradecyl)-2-ethylpyridinium, 1-(1-hexadecyl)-2-ethylpyridinium, 1,2-dimethyl-5-ethylpyridinium, 1,5-diethyl-2-methylpyridinium, 1-(1-butyl)-2-methyl-3-ethylpyridinium, 1-(1-hexyl)-2-methyl-3-ethylpyridinium, 1-(1-octyl)-2-methyl-3-ethylpyridinium, 1-(1-dodecyl)-2-methyl-3-ethylpyridinium, 1-(1-tetradecyl)-2-methyl-3-ethylpyridinium, 1-(1-hexadecyl)-2-methyl-3-ethylpyridinium, 1-methylimidazolium, 1-ethylimidazolium, 1-(1-butyl)-imidazolium, 1-(1-octyl)imidazolium, 1-(1-dodecyl)-imidazolium, 1-(1-tetradecyl)imidazolium, 1-(1-hexadecyl)imidazolium, 1,3-dimethylimidazolium, 1-ethyl-3-methylimidazolium, 1-(1-butyl)-3-methylimidazolium, 1-(1-hexyl)-3-methylimidazolium, 1-(1-octyl)-3-methylimidazolium, 1-(1-dodecyl)-3-methylimidazolium, 1-(1-tetradecyl)-3-methylimidazolium, 1-(1-hexadecyl)-3-methylimidazolium, 1,2-dimethylimidazolium, 1,2,3-trimethylimidazolium, 1-ethyl-2,3-dimethylimidazolium, 1-(1-butyl)-2,3-dimethylimidazolium, 1-(1-hexyl)-2,3-dimethylimidazolium and 1-(1-octyl)-2,3-dimethylimidazolium, 1,4-dimethylimidazolium, 1,3,4-trimethylimidazolium, 1,4-dimethyl-3-ethylimidazolium, 3-butylimidazolium, 1,4-dimethyl-3-octylimidazolium, 1,4,5-trimethylimidazolium, 1,3,4,5-tetramethylimidazolium, 1,4,5-trimethyl-3-ethylimidazolium, 1,4,5-trimethyl-3-butylimidazolium and 1,4,5-trimethyl-3-octylimidazolium.

[Y]ⁿ⁻ is an anion selected from among
the group of sulfates, sulfites and sulfonates of the general formulae: SO₄²⁻, HSO₄⁻, SO₃²⁻, HSO₃—, R^{A#}OSO₃—, R^{B#}SO₃—;
the group of phosphates of the general formulae PO₄³⁻, HPO₄²⁻, H₂PO₄—, R^{a}PO₄²⁻, HR^{A#}PO₄—, R^{A#}R^{B#}PO₄—;
the group of phosphonates and phosphinates of the general formulae: R^{A#}HPO₃—, R^{A#}R^{B#}PO₂⁻, R^{A#}R^{B#}PO₃—;
the group of phosphites of the general formulae: PO₃³⁻, HPO₃²⁻, H₂PO₃—, R^{a}PO₃²⁻, R^{A#}HPO₃—, R^{A#}R^{B#}PO₃—;
the group of phosphonites and phosphinites of the general formulae: R^{A#}R^{B#}PO₂—, R^{A#}HPO₂—, R^{A#}R^{B#}PO—, R^{A}HPO—;
the group of carboxylic acids of the general formula: R^{A#}COO—;
NO₃—.
Here, R^{A#} and R^{B#} are each, independently of one another, hydrogen, C₁-C₃₀-alkyl, C₂-C₁₈-alkyl which may optionally be interrupted by one or more nonadjacent oxygen and/or sulfur atoms and/or one or more substituted or unsubstituted imino groups, C₆-C₁₄-aryl, C₅-C₁₂-cycloalkyl or a five- or six-membered, oxygen-, nitrogen- and/or sulfur-comprising heterocycle, where two of them may also together form an unsaturated, saturated or aromatic ring which may optionally be interrupted by one or more oxygen and/or sulfur atoms and/or one or more unsubstituted or substituted imino groups, where the radicals mentioned may each be additionally substituted by functional groups, aryl, alkyl, aryloxy, alkyloxy, halogen, heteroatoms and/or heterocycles.
Here, C₁-C₁₈-alkyl which may optionally be substituted by functional groups, aryl, alkyl, aryloxy, alkyloxy, halogen, heteroatoms and/or heterocycles is, for example, methyl, ethyl, propyl, isopropyl, n-butyl, sec-butyl, tert-butyl, pentyl, hexyl, heptyl, octyl, 2-ethylhexyl, 2,4,4-trimethylpentyl, decyl, dodecyl, tetradecyl, hexadecyl, octadecyl, 1,1-dimethylpropyl, 1,1-dimethylbutyl, 1,1,3,3-tetramethylbutyl, benzyl, 1-phenylethyl, α,α-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, CₘF_{2(m-a)+(1- b)}H_{2a+b} where m is from 1 to 18, 0≦a≦m and b=0 or 1 (for example CF₃, C₂F₅, CH₂CH₂—C₍ₘ₋₂₎F₂₍ₘ₋₂₎₊₁, C₆F₁₃, C₈F₁₇, C₁₀F₂₁, C₁₂F₂₅), chloromethyl, 2-chloroethyl, trichloromethyl, 1,1-dimethyl-2-chloroethyl, 2-methoxycarbonethyl, 2-ethoxycarbonylethyl, 2-butoxycarbonylpropyl, 1,2-di-(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, trichloromethyl, trifluoromethyl, 1,1-dimethyl-2-chloroethyl, 2-methoxyisopropyl, 2-ethoxyethyl, butylthiomethyl, 2-dodecylthioethyl, 2-phenylthioethyl, 2,2,2-trifluoroethyl, 2-hydroxyethyl, 2-hydroxypropyl, 3-hydroxypropyl, 4-hydroxybutyl, 6-hydroxyhexyl, 2-aminoethyl, 2-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl or 6-ethoxyhexyl.
C₂-C₁₈-alkyl which may optionally be interrupted by one or more nonadjacent oxygen and/or sulfur atoms and/or one or more substituted or unsubstituted imino groups is, for example, 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxa-nonyl, 14-hydroxy-5,10-oxatetradecyl, 5-methoxy-3-oxapentyl, 8-methoxy-3,6-dioxa-octyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-oxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 1 4-ethoxy-5,10-oxatetradecyl.
If two radicals form a ring, these radicals can together form as fused-on building block, for example, 1,3-propylene, 1,4-butylene, 2-oxa-1,3-propylene, 1-oxa-1,3-propylene, 2-oxa-1,3-propenylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.
The number of nonadjacent oxygen and/or sulfur atoms and/or imino groups is in principle not subject to any restrictions or is automatically restricted by the size of the radical or the cyclic building block. In general, there will be no more than 5 in the respective radical, preferably no more than 4 and very particularly preferably no more than 3. Furthermore, there is generally at least one carbon atom, preferably at least two carbon atoms, between any two heteroatoms.
Substituted and unsubstituted imino groups can be, for example, imino, methylimino, isopropylimino, n-butylimino or tert-butylimino.
For the purposes of the present invention, the term “functional groups” refers, for example, to the following: carboxy, carboxamide, hydroxy, di-(C₁-C₄-alkyl)amino, C₁-C₄-alkyloxycarbonyl, cyano or C₁-C₄-alkoxy. Here, C₁ to C₄-alkyl is methyl, ethyl, propyl, isopropyl, n-butyl, sec-butyl or tert-butyl.
C₆-C₁₄-aryl which may optionally be substituted by functional groups, aryl, alkyl, aryloxy, alkyloxy, halogen, heteroatoms and/or heterocycles is, for example, phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl.
C₅-C₁₂-cycloalkyl which may optionally be substituted by functional groups, aryl, alkyl, aryloxy, halogen, heteroatoms and/or heterocycles is, for example, cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl or a saturated or unsaturated bicyclic system such as norbornyl or norbornenyl.
A five- or six-membered, oxygen-, nitrogen- and/or sulfur-comprising heterocycle is, for example, furyl, thiophenyl, pyryl, pyridyl, indolyl, benzoxazolyl, dioxolyl, dioxyl, benzimidazolyl, benzthiazolyl, dimethylpyridyl, methylquinolyl, dimethylpyryl, methoxyfuryl, dimethoxypyridyl, difluoropyridyl, methylthiophenyl, isopropylthiophenyl or tert-butylthiophenyl.
Preferred anions are selected from the group of carboxylic acids, the group of sulfates, sulfites and sulfonates and the group of phosphates, in particular from the group of sulfates, the group of carboxylic acids and the group of sulfonates and NO₃—.
Particular preference is given to SO₄²⁻, HSO₄—; C₁-C₄-alkyl-COO—, in particular acetate and propionate, preferable acetate; C₁-C₄-halogenalkyl-COO—, in particular trifluoroacetate or perfluoropropionate, preferably trifluoroacetate; C₁-C₄₋alkyl-SO₃—, preferably methylsulfonate, ethylsulfonate or butylsulfonate, preferably methylsulfonate; or C₁-C₄-halogenalkyl-SO₃—, preferably trifluoromethylsulfonate or perfluoroethylsulfonate, preferably trifluoromethylsulfonate.
Very particular preference is given to SO₄²⁻, HSO₄—, acetate and methylsulfonate.

In a preferred embodiment, ionic liquids of the formula II in which the variable [A]⁺ is as defined under the ionic liquids of the formula I and X— is chloride or bromide, preferably chloride, are used. The preferences for [A]⁺ apply in an analogous way.

In a preferred embodiment, amines of the formula V in which the radicals
R¹ to R³ are each, independently of one another, C₁-C₁₈-alkyl; or
R¹ and R² are together 1,5-pentylene or 3-oxa-1,5-pentylene and R³ is C₁-C₁₈-alkyl, 2-hydroxyethyl or 2-cyanoethyl,
   and the number of carbon atoms of the radicals R¹, R² and R³ together is at least 10 but not more than 30 are used.

In a further embodiment, amines of the formula V in which
one of the radicals R¹ to R³ is hydrogen and the other two radicals are each, independently of one another, C₁-C₁₈-alkyl;
   and the number of carbon atoms of the radicals R² and R³ together is at least 10 but not more than 30 are used.

In a particular embodiment, use is made of diethylhexylamine, diethyloctylamine, diethyl(2-ethylhexyl)amine, di-n-propylbutylamine, di-n-propyl-n-pentylamine, di-n-propylhexylamine, di-n-propyloctylamine, di-n-propyl(2-ethylhexyl)amine, diisopropylbutylamine, diisopropylpentylamine, diisopropylhexylamine, diisopropyloctylamine, diisopropyl-(2-ethylhexyl)amine, di-n-butylethylamine, di-n-butyl-n-propylamine, di-n-butyl-n-pentylamine, di-n-butylhexylamine, di-n-butyloctylamine, di-n-butyl-(2-ethylhexyl)amine, dioctylamine, diethylhexylamine, dinonylamine, tripentylamine, trihexylamine, trioctylamine, trinonylamine, tridecylamine, methyldi(2-ethylhexyl)amine, ethyldi(2-ethylhexyl)amine, propyldi(2-ethylhexyl)amine, butyldi(2-ethylhexyl)amine, isopropyldi(2-ethylhexyl)amine, isobutyldi(2-ethylhexyl)amine or tris(2-ethylhexyl)amine, preferably diethylhexylamine, diethyloctylamine, diethyl(2-ethylhexyl)amine, di-n-propylbutylamine, di-n-propyl-n-pentylamine, di-n-propylhexylamine, di-n-propyloctylamine, di-n-propyl(2-ethylhexyl)amine, di-isopropylbutylamine, diisopropylpentylamine, diisopropylhexylamine, diisopropyloctylamine, diisopropyl-(2-ethylhexyl)amine, di-n-butylethylamine, di-n-butyl-n-propylamine, di-n-butyl-n-pentylamine, di-n-butylhexylamine, di-n-butyloctylamine, di-n-butyl-(2-ethylhexyl)amine, dioctylamine, diethylhexylamine, dinonylamine, tripentylamine, trihexylamine, trioctylamine, trinonylamine or tridecylamine.

The present invention is illustrated by the following examples.

### EXAMPLE 1

13.11 g of 1-butyl-3-methylimidazolium chloride (75 mmol), 7.247 g (75 mmol) of methanesulfonic acid and 42.11 g of trihexylamine (150 mmol) in 42.11 g of toluene were heated to 40° C. After stirring for about 1 hour, the mixture was cooled to room temperature and the phases which formed were separated. The lower phase is dried at 80° C. and a pressure of 10 mbar. This gave 14.4 g of butyl-3-methylimidazolium methylsulfonate (which comprised 3.3% by weight of chloride).

### EXAMPLE 2

8.74 g of 1-butyl-3-methylimidazolium chloride (50 mmol), 3.00 g (50 mmol) of acetic acid (99%) and 28.00 g of trihexylamine (100 mmol) in 28 g of toluene were heated to 40° C. After stirring for about 1 hour, the mixture was cooled to room temperature and the phases which formed were separated. The lower phase is dried at 80° C. and a pressure of 10 mbar. This gave 7.0 g of butyl-3-methylimidazolium acetate.

1: A process for preparing ionic liquids of the formula (I)
[A]⁺ₙ[Y]ⁿ⁻ (I)
where
[A]⁺ is a quaternary nitrogen heterocycle cation, oxonium cation, sulfonium cation or phosphonium cation;
n is 1,2, 3 or 4; and
[Y]ⁿ⁻ is an anion selected from among
the group of sulfates, sulfites and sulfonates,
the group of phosphates,
the group of phosphonates and phosphinates,
the group of phosphites,
the group of phosphonites and phosphinites,
the group of carboxylic acids and
NO₃⁻;
by reacting an ionic liquid of the formula (II)
[A]⁺[X]⁻ (II)
where
[A]⁺ is as defined above; and
[X]⁻ is fluoride, chloride, bromide or iodide;
with an acid of the formula III
H⁺ₙ[Y]ⁿ⁻ (III)
where n and [Y]ⁿ⁻ are as defined above;
characterized in that the resulting acid of the formula IV
H⁺[X]⁻ (IV)
where [X]⁻ is as defined above;
is scavenged by means of an amine of the formula V
NR¹R¹R³ (V)
where
R¹, R² and R³ are each, independently of one another, hydrogen or C₁-C₂₀-alkyl, which may optionally be substituted, with the number of carbon atoms of the radicals R¹, R² and R³ together being at least 10;
and the resulting ammonium halide of the formula VI
[HNR¹R²R³]⁺[X]⁻ (VI)
where the radicals X, R¹, R² and R³ are as defined above;
is separated off.

2: The process according to claim 1, wherein [A]⁺ in the formulae I and II is a cation selected from among the compounds of the formulae (Ia) to (Iv), 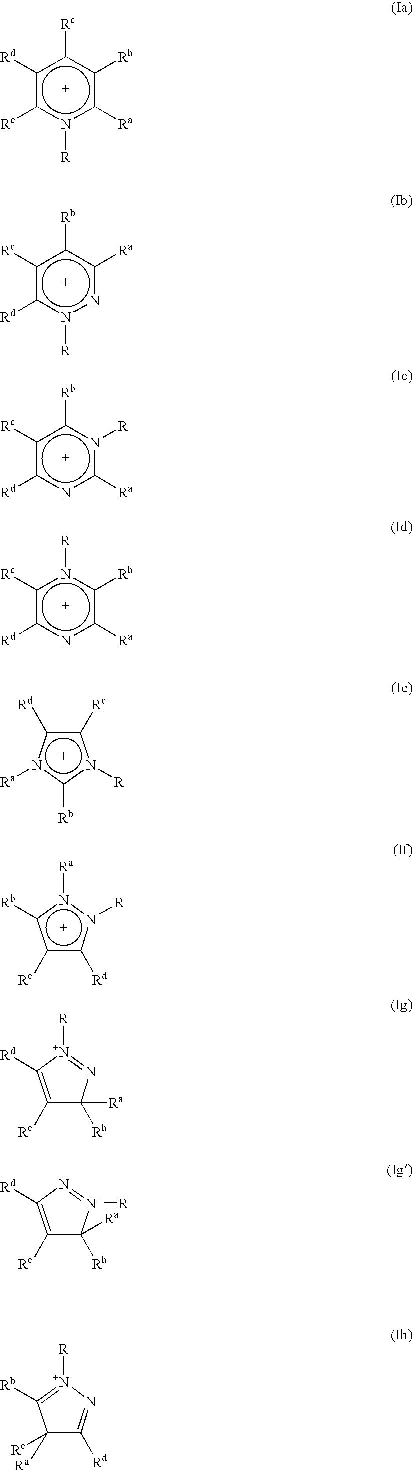 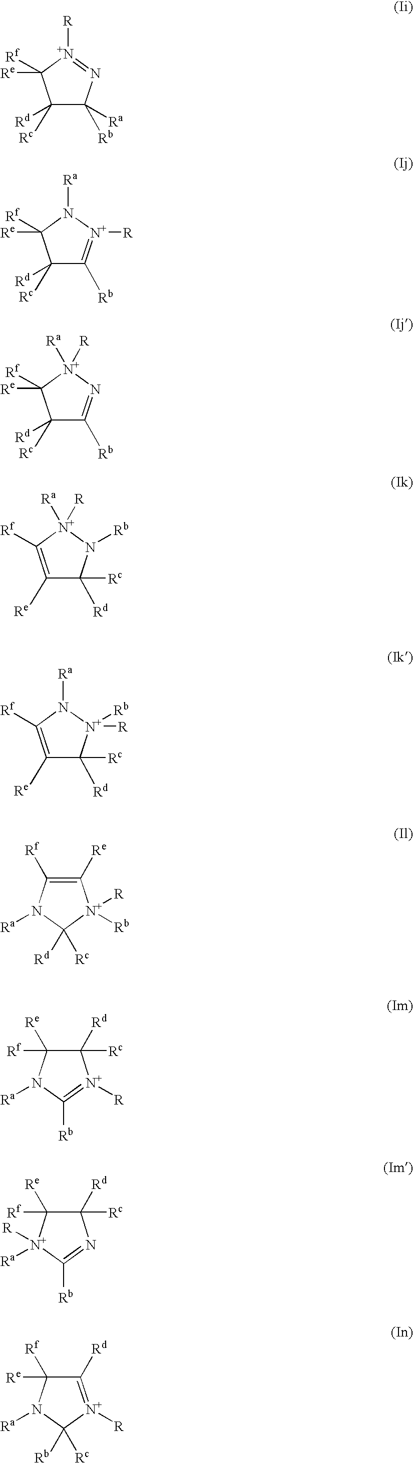 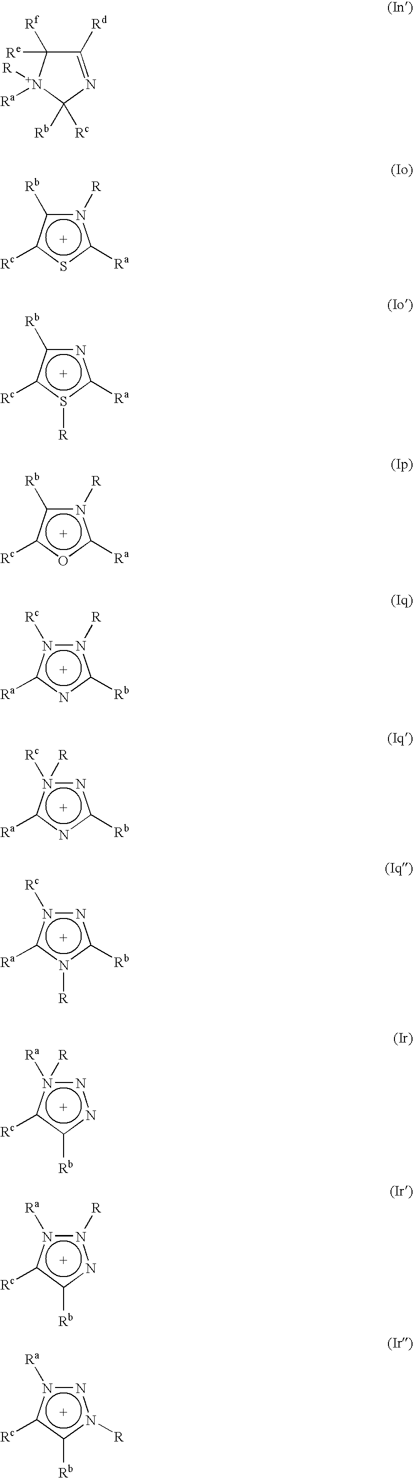 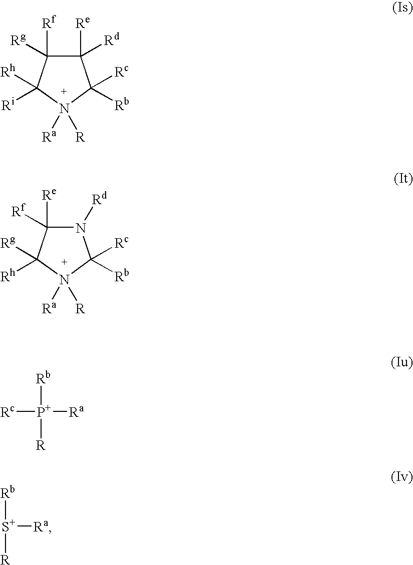
where
the radical R is hydrogen or a carbon-containing organic, saturated or unsaturated, acyclic or cyclic, aliphatic, aromatic or araliphatic radical which has from 1 to 20 carbon atoms and may be unsubstituted or be interrupted or substituted by from 1 to 5 heteroatoms or functional groups; and
the radicals R^{a} to Rⁱ are each, independently of one another, hydrogen or a carbon-containing organic, saturated or unsaturated, acyclic or cyclic, aliphatic, aromatic or araliphatic radical which has from 1 to 20 carbon atoms and may be unsubstituted or be interrupted or substituted by from 1 to 5 heteroatoms or functional groups, where the radicals R^{a} to Rⁱ which are bound to a carbon atom (and not to a heteroatom) in the formulae (I) above can additionally be halogen or a functional group;
or
two adjacent radicals from the group consisting of R^{a} to Rⁱ may together also form a divalent, carbon-containing organic, saturated or unsaturated, acyclic or cyclic, aliphatic, aromatic or araliphatic radical which has from 1 to 30 carbon atoms and may be unsubstituted or be interrupted or substituted by from 1 to 5 heteroatoms or functional groups.

3: The process according to claim 1, wherein [Y]ⁿ⁻ in the formulae I and III is an anion selected from among
the group of sulfates, sulfites and sulfonates of the general formulae: SO₄²⁻, HSO₄⁻, SO₃²⁻, HSO₃⁻, R^{A#}OSO₃⁻, R^{B#}SO₃⁻;
the group of phosphates of the general formulae PO₄³⁻, HPO₄²⁻, H₂PO₄⁻, R^{a}PO₄²⁻, HR^{A#}PO₄⁻, R^{A#}R^{B#}PO₄⁻;
the group of phosphonates and phosphinates of the general formulae: R^{A#}HPO₃⁻, R^{A#}R^{B#}PO₂⁻, R^{A#}R^{B#}PO₃⁻;
the group of phosphites of the general formulae: PO₃³⁻, HPO₃²⁻, H₂PO₃⁻, R^{a}PO₃²⁻, R^{A#}HPO₃⁻, R^{A#}R^{B#}PO₃⁻;
the group of phosphonites and phosphinites of the general formulae: R^{A#}R^{B#}PO₂⁻, R^{A#}HPO₂⁻, R^{A#}R^{B#}PO⁻, R^{A}HPO⁻;
the group of carboxylic acids of the general formula: R^{A#}COO⁻;
where R^{A#} and R^{B#} are each, independently of one another, hydrogen, C₁-C₃₀-alkyl, C₂-C₁₈-alkyl which may optionally be interrupted by one or more non-adjacent oxygen and/or sulfur atoms and/or one or more substituted or unsubstituted imino groups, C₆-C₁₄-aryl, C₅-C₁₂-cycloalkyl or a five- or six-membered, oxygen-, nitrogen- and/or sulfur-comprising heterocycle, where two of them may also together form an unsaturated, saturated or aromatic ring which may optionally be interrupted by one or more oxygen and/or sulfur atoms and/or one or more unsubstituted or substituted imino groups, where the radicals mentioned may each be additionally substituted by functional groups, aryl, alkyl, aryloxy, alkyloxy, halogen, heteroatoms and/or heterocycles.

4: The process according to claim 1, wherein [X]⁻ in the formulae II and IV is chloride or bromide.

5: The process according to claim 2, wherein [A]⁺ is a cation selected from the group consisting of the compounds Ia, Ie, If, Ig, Ig′, Ih, Ii, Ij, Ij′, Ik, Ik′, Il, Im, Im′, In and In′.

6: The process according to claim 2, wherein [A]⁺ is a cation selected from the group consisting of the compounds Ia, Ie and If.

7: The process according to claim 1, wherein [Y]ⁿ⁻ in the formulae I and III is an anion selected from the group consisting of SO₄²⁻, HSO₄—; C₁-C₄-alkyl-COO—, C₁-C₄-haloalkyl-COO⁻, C₁-C₄-alkyl-SO₃⁻ and C₁-C₄-haloalkyl-SO₃⁻.

8: The process according to claim 1, wherein the ammonium halide of the formula VI is separated off from the ionic liquid of the formula I by liquid/liquid phase separation.

9: The process according to claim 1, wherein the amine of the formula V is recovered from the ammonium halide of the formula VI which has been separated off.

10: The process according to claim 9, wherein the ammonium halide of the formula VI is reacted with a strong base and the amine of the formula V is separated off.

11: The process according to claim 9, wherein the amine of the formula V is fed continuously into a mixture of ionic liquid of the formula II and acid of the formula III, the ammonium halide of the formula VI which is formed is continuously separated off and the amine of the formula V recovered from the ammonium halide is recirculated to the reaction mixture.

12: The process according to claim 11, wherein both the amine of the formula V and the mixture of ionic liquid of the formula II and acid of the formula III are fed continuously to a mixing and separation apparatus.
